(19) United States
(12) Patent Application Publication
(10) Pub. No.: US 2025/0000470 A1
(43) Pub. Date: Jan. 2, 2025

(54) COMPUTER-IMPLEMENTED METHOD FOR OPERATING AN X-RAY DEVICE, X-RAY DEVICE, COMPUTER PROGRAM AND ELECTRONICALLY READABLE DATA CARRIER

(71) Applicant: Siemens Healthineers AG, Forchheim (DE)

(72) Inventors: Magdalena HERBST, Erlangen (DE); Marcel BEISTER, Erlangen (DE); Thomas WEBER, Hausen (DE); Ludwig RITSCHL, Buttenheim (DE)

(73) Assignee: Siemens Healthineers AG, Forchheim (DE)

(21) Appl. No.: 18/756,119

(22) Filed: Jun. 27, 2024

(30) Foreign Application Priority Data
Jun. 29, 2023 (DE) 10 2023 206 155.4

(51) Int. Cl. A61B 6/50; A61B 6/02; A61B 6/04; A61B 6/42; A61B 6/58

(52) U.S. Cl. CPC A61B 6/502; A61B 6/025; A61B 6/0435; A61B 6/4208; A61B 6/589

(57) ABSTRACT

A computer-implemented method for operating an X-ray device during an imaging process on an examination object, comprises: ascertaining extension information from measurement data of at least one measuring device, the extension information describing extension of the examination object in at least one spatial direction; and parameterizing a reconstruction process based on the extension information; wherein an image dataset covering a reconstruction area is reconstructed in the reconstruction process from projection images of the examination object, which are captured in different projection geometries of an X-ray emitter relative to an X-ray detector of the X-ray device.

### CROSS-REFERENCE TO RELATED APPLICATION(S)

The present application claims priority under 35 U.S.C. § 119 to German Patent Application No. 10 2023 206 155.4, filed Jun. 29, 2023, the entire contents of which is incorporated herein by reference.

### FIELD

One or more example embodiments of the present invention relate to a computer-implemented method for operating an X-ray device during an imaging process on an examination object, wherein an image dataset covering a reconstruction area is reconstructed in a reconstruction process from projection images of the examination object which are captured in different projection geometries of an X-ray emitter in relation to an X-ray detector of the X-ray device. One or more example embodiments of the present invention also relate to an X-ray device, a computer program, a non-transitory computer-readable medium and/or electronically readable data carrier.

### BACKGROUND

X-ray devices are now established in the prior art in the medical field. Tomosynthesis as a sub-area of X-ray imaging aims to obtain three-dimensional information with little effort by capturing projection images from different projection geometries from which an image dataset can be reconstructed, for example one or more slice images (sectional images) from a capturing area of the examination object. For this purpose, it can, for example, be provided that the X-ray emitter is moved relative to the X-ray detector and, for example, is brought into different angular positions, thus realizing a specific capturing trajectory of the X-ray emitter, while the X-ray detector remains (at least substantially) stationary. This is, for example, used in mammography. In addition, it is known to move an X-ray emitter and an X-ray detector in parallel in a capturing direction so that different beam directions occur due to the cone beam geometry of the X-ray emitter through respective voxels to be resolved and provide depth information. Such a technique can, for example, be used in radiography and/or fluoroscopy devices.

While techniques are known that use tomosynthesis capturing of this kind for an extended field of view when reconstructing a specific slice within the examination object, in conventional tomosynthesis, for example mammography, usually a plurality of slice images or sectional images are calculated, in this example through the female breast, which in particular at least substantially follow one another in the direction of a central beam of a central projection geometry or are “stacked”. This direction of the central beam can then also be referred to as the slice direction.

For a person making the diagnosis, for example a diagnostic radiologist, usually only slice images that map the examination object are relevant, but on the other hand, they also want to view the examination object in its entirety. In other words, the reconstruction of slices in one slice direction should, for example, cover the entire depth of the examination object depicted. For this purpose, it is, for example, known that a fixed extension area to be reconstructed in the slice direction can be specified in dependence on a capturing protocol or selected based on user input. This is imprecise and not tailored to a current examination object, for example a patient.

It has also already been proposed in the prior art that a reconstruction area to be reconstructed be estimated based on images, for example as part of a first rough reconstruction. However, this approach is quite complex and requires additional calculations, in particular additional reconstructions, and reliable segmentation or detection methods.

### SUMMARY

One or more embodiments of the present invention are based on at least an object of ascertaining and using extension information that is specific to an examination object in an easy-to-implement and reliable manner for consideration in the reconstruction.

At least this object is achieved according to embodiments of the present invention by a computer-implemented method, an X-ray device, a computer program and an electronically readable data carrier as claimed.

With a method of the type mentioned in the introduction, it is provided according to an embodiment of the present invention that
extension information describing the extension of the examination object in at least one spatial direction is ascertained from measurement data of at least one measuring device and/or means and
the reconstruction process is parameterized in dependence on the extension information.

Herein, the projection images are preferably captured in cone beam geometry in order to be able to obtain different beam paths in a simplified manner when at least the X-ray emitter is moved. The extension information is ascertained in the coordinate system of the X-ray device in which the reconstruction also takes place. It indicates the boundaries of the examination object in the at least one spatial direction. If, for example, the spatial direction is the direction of a central beam of the X-ray device in a projection geometry, in particular at least one central projection geometry along a capturing trajectory of the X-ray emitter, a boundary on the X-ray emitter side (“upper end”) and a boundary on the X-ray detector side (“lower end”) of the examination object are known from the extension information.

Therefore, according to an embodiment of the present invention, it is proposed that the extension information is not ascertained from a specification, an estimated user input or the image data, for example the projection images and/or a reconstruction result, but by an additional measuring device and/or means which is independent of the capturing arrangement (comprising an X-ray emitter and X-ray detector) and which preferably automatically detects and provides the measurement data, whereupon the extension information can be ascertained automatically and used automatically.

Therefore, according to an embodiment of the present invention, it is proposed that the boundaries of the examination object be ascertained in at least one spatial direction (of interest), particularly preferably completely automatically, using a measuring device and be used to parameterize the reconstruction, i.e., when selecting at least one reconstruction parameter. In this way, the extension information describing the extension of the examination object, in particular a patient, in the spatial direction in the coordinate system of the X-ray device can be ascertained reliably and precisely but nevertheless in a simple manner, in particular without complex image evaluation. Herein, it should already be noted at this point that it is not mandatory for both boundaries to be derived from the measurement data of the measuring device, instead it is also possible to identify a boundary in another way, for example by lying the examination object on a component of the X-ray device. If the extension information is determined completely automatically, manual (correction) steps can be omitted.

In order to be able to ascertain the extension information in the coordinate system of the X-ray device, the measuring device is preferably registered, or can be registered, with the coordinate system of the X-ray device. For this purpose, the measuring device can, for example, be positioned in a defined manner on or at least relative to the X-ray device.

Particularly expediently, the present invention can be used in the context of tomosynthesis and so it can be provided that the imaging process is a tomosynthesis process and/or that the different projection geometries are established either by parallel displacement of the X-ray emitter and the X-ray detector or by movement of the X-ray emitter relative to the fixed X-ray detector. Herein, the latter variant can in particular comprise moving the X-ray emitter along a circular path or moving the X-ray emitter along a linear capturing trajectory while tilting and/or adapting the collimation of the X-ray emitter. As mentioned, in the case of linear capturing trajectories, different beam directions are created by voxels of interest due to the cone beam geometry used (often also referred to as fan beam geometry).

Herein, the X-ray device can be a radiography and/or fluoroscopy device. In this case, a tomosynthesis process can expediently be achieved in that the X-ray emitter and the X-ray detector are moved parallel to one another in the capturing direction, wherein cone beam geometry or fan beam geometry is used. However, radiography and/or fluoroscopy devices have already been proposed with which further degrees of freedom of movement are possible and therefore it is also possible to realize more complex capturing geometries in order to perform tomosynthesis. In particular, it is in principle also conceivable in the context of the present invention for a C-arm device to be used as an X-ray device with which a capturing trajectory can be realized by rotating the C-arm. However, it is preferable to use simply designed radiography and/or fluoroscopy devices that are extended by expedient convenient tomosynthesis functions. According to the present invention, these can also be improved in a simple manner with respect to ascertaining extension information.

However, it can also be provided that the X-ray device is a mammography device. A mammography device can have an object holder for the breast to be captured, which is, for example, directly bounded on one side by the X-ray detector (or the cover thereof), so that a boundary there is known in the system. In the case of a compression device, an adjustable compression plate can be provided on the other side and its position can be detected via the measuring device. To ascertain the extension in other spatial directions or if no compression plate is used, particularly advantageously a different type of measuring device can be used, for example one of the measuring device described below.

In a particularly advantageous development of one or more embodiments of the present invention, it can be provided that at least one distance sensor and/or at least one camera measuring in three dimensions is used as the measuring device. The distance sensor can, for example, be a radar sensor. However, cameras measuring in three dimensions (3D cameras) are particularly preferred, wherein different specific embodiments, such as those known in principle in the prior art, can be used in the context of the present invention. For example, a terahertz camera and/or a TOF camera (time-of-flight camera) and/or a stereoscopic camera can be used as a camera measuring in three dimensions (3D-camera). Here, the use of cameras that also provide optical image information has the advantage that it is possible to use image processing algorithms that are already known, in order, for example, to be able to detect the examination object.

It can generally be said that particularly preferably the measurement data can be evaluated to form a model that at least partially describes the examination object, in particular the patient, specifically its surface. This model can then be used to derive at least one boundary of the examination object in the at least one spatial direction. Expediently, when creating such a model, it is also possible to take account of at least one item of prior knowledge information, which is, for example, provided by an information system (such as a radiology information system), for example prior knowledge information relating to the height and/or weight and/or gender and/or orientation of the examination object. This can, for example, enable the selection of a suitable starting point for creating the model.

In particular, if the measuring device or one of the at least one measuring device is to be used primarily to ascertain a specific boundary, for example an “upper boundary”, it can be provided that at least one of the at least one distance sensor and/or at least one of the at least one camera is attached to the X-ray emitter or to a component of the X-ray device which is firmly connected to the X-ray emitter, in particular to a collimator device. An arrangement on or adjacent to the X-ray emitter in a fixed spatial relationship thereto makes it particularly easy to ascertain the distance between the X-ray emitter and the facing boundary of the examination object (source-object distance, SOD). For example, here, the measuring device can detect a distance to the impact area of a central beam in a targeted manner. It is, for example, conceivable to attach a distance sensor and/or a 3D camera to a collimator device.

However, in preferred exemplary embodiments, it can also be provided that, additionally or alternatively, a plurality of distance sensors and/or cameras arranged at different positions are used, the measurement data of which is evaluated at least in the spatial direction to form a model describing the boundaries of the examination object. For example, it is possible to use measuring device, in particular cameras measuring in three dimensions, which are distributed on the X-ray device and/or in a room in which the X-ray device is arranged in particular in such a way that the complete (visible or viewable) surface of the examination object can be measured and thus a corresponding, as complete as possible, three-dimensional model of the examination object can be ascertained. Corresponding techniques for the three-dimensional measurement of objects and for creating corresponding models, in particular, as mentioned, taking into account prior knowledge information, are in principle known in the prior art and can also be used profitably in the context of the present invention.

It can be provided that the extension information is ascertained using user input. Here, it is, for example, in principle conceivable for the aforementioned prior knowledge information also to be obtained via user input. However, exemplary embodiments are also conceivable in which the user input comprises at least part of the measurement data. This is in particular expedient if at least one of the at least one measuring device is to be operated manually, at least partially. For example, a measuring tape, which is arranged with the X-ray emitter and/or the X-ray detector to measure a distance, can be provided for the user as at least one of the at least one measuring device. Such a measuring tape as at least one of the at least one measuring device can, for example, be arranged on the X-ray emitter or a component of the X-ray device that is permanently connected to the X-ray emitter, in particular to a collimator device, so that the distance to the side of the examination object facing the X-ray emitter can be measured manually and entered as user input. This is in particular also conceivable with a free-standing patient as the examination object. Generally, for example, user input can be entered on an input device of the X-ray device, in particular on a so-called touch user interface (touch UI).

However, automatic measuring device and/or means, in particular cameras measuring in three dimensions, are preferred to the use of manually operated measuring device.

As already indicated, in an expedient development of the present invention, it can be provided that a known position and/or a position that can be detected via at least one of the at least one measuring device of an examination object-side boundary of the component is used as at least one boundary of the extension of the examination object in the spatial direction when the examination object is mounted on a component of the X-ray device. As already mentioned, such a constellation is known in some imaging processes, for example in mammography when the breast to be captured rests on the X-ray detector, and/or in other tomosynthesis examinations in which the patient lies on a patient bench and/or is seated. In such situations, a boundary of the examination object is known by the position of the corresponding component, which is known to the control device of the X-ray device. The component on which the examination object is arranged can, for example, be the patient bench (for example a patient table top), the X-ray detector (or its cover) and the like.

In the context of the present invention, different variants for the specific use of the extension information for the reconstruction process are conceivable.

For example, on the one hand, it can be provided that the reconstruction area in the spatial direction is restricted to the extent of the examination object. For example, it can be achieved from the outset that reconstruction only takes place where the examination object is also present, but the entire examination object is nevertheless covered. Generally speaking, the spatial direction can be a slice direction and/or, for at least one projection geometry, the direction of a central beam of the X-ray emitter. For example, when ascertaining slice images (sectional images) as a slice image stack in a slice direction corresponding to the direction of the central beam at least for a central projection geometry, in other words a fluoroscopy direction, the slices can be restricted to those that cover the examination object as precisely as possible. On the other hand, complete coverage of the examination object ensures that the image dataset contains image data for the entire examination object in the spatial direction.

However, particularly expediently, the extension information can also be used in so-called slot scanning techniques or other capturing techniques in which, in particular in a radiography and/or fluoroscopy device, the X-ray emitter and the X-ray detector are moved in parallel along the examination object in order to capture a larger area due to the cone beam geometry while additionally obtaining 3D information.

It can therefore be provided that, in the imaging process, an image area which is extended with respect to the detection area of the X-ray device is captured in a capturing direction perpendicular to the spatial direction by a parallel movement of the X-ray emitter and the X-ray detector in the capturing direction and a focal area of the reconstruction is selected in dependence on the extension information. Here, the image area can, for example, cover at least ten times the detection area. This is, for example, expedient if the head and the torso are to be captured in their entirety, for example to examine the spine, and/or a leg is to be captured in its entirety. For example, this is often referred to as a so-called body scan. It is known to reconstruct at least one, usually somewhat thicker, slice, which may, for example, contain a bone or a bone arrangement of interest, from the projection images. Herein, the choice of focal depth is extremely important to ensure that the structure of interest of the examination object to be mapped, for example the bone or the bone arrangement, is depicted with sufficient sharpness. The extension information can be used to adjust the focal area, in particular automatically, with improved accuracy. Here, it can be specifically provided that the focal area is selected in dependence on the position of a structure to be mapped in the spatial direction in the examination object. Herein, for example, the position of the structure to be mapped within the extension can, for example, be specified as part of the prior knowledge information.

Preferably, the projection images can be captured with collimation in the capturing direction, in particular as a slot scan. Here, collimation can reduce the detection area in the capturing direction by at least a factor of five, in particular at least by a factor of ten. Nevertheless, the cone beam geometry leaves a cone or fan that allows different beam directions through voxels of interest and thus allows three-dimensional information to be ascertained. As a result, in particular the focal area also remains of particular importance.

In addition to the method, one or more embodiments of the present invention also relate to an X-ray device having an X-ray emitter, an X-ray detector, at least one measuring device and a control device, which is embodied to execute the method according to embodiments of the present invention. All statements relating to the method according to embodiments of the present invention can be transferred analogously to the X-ray device according to embodiments of the present invention and therefore the advantages already mentioned can also be obtained with this device.

In particular, the control device can contain at least one processor and/or at least one memory device and/or means. Functional units for performing steps of the method according to embodiments of the present invention can be formed from hardware and/or software components. The control device can, for example, comprise a capturing unit that controls the capturing operation of the X-ray device and which can also control the imaging process for capturing the projection images, in particular in a tomosynthesis process. Measurement data of the at least one measuring device can be evaluated in an ascertaining unit in order to ascertain the extension information. The extension information can be used in a reconstruction unit which is embodied to perform the reconstruction process in order to parameterize the reconstruction process. Obviously, further functional units are also conceivable. The X-ray device can, for example, be a radiography and/or fluoroscopy device and/or a mammography device.

A computer program according to an embodiment of the present invention can be loaded directly into a memory device of a control device of an X-ray device and has program device and/or means which cause the control device to perform the steps of a method according to embodiments of the present invention when the computer program is executed on the control device. The computer program can be stored on a non-transitory computer-readable medium and/or an electronically readable data carrier according to embodiments of the present invention which therefore has control information stored thereon which comprises at least one computer program according to embodiments of the present invention and is embodied such that, when the data carrier is used in a control device of an X-ray device, this is embodied to perform a method according to embodiments of the present invention.

### BRIEF DESCRIPTION OF THE DRAWINGS

Further advantages and details of the present invention emerge from the exemplary embodiments described below and from the drawings, in which:

FIG. 1 shows a flow chart of an exemplary embodiment of the method according to the present invention,

FIG. 2 shows a possible positioning of an examination object and a measuring device in a first capturing situation,

FIG. 3 shows a possible positioning of an examination object and measuring device in a second capturing situation,

FIG. 4 shows a possible positioning of an examination object and measuring device in a third capturing situation,

FIG. 5 shows a covering of an examination object by slices,

FIG. 6 shows a focal area during a slot scan,

FIG. 7 shows a functional schematic outline of an X-ray device according to an embodiment of the present invention, and

FIG. 8 shows the functional structure of a control device of the X-ray device.

### DETAILED DESCRIPTION

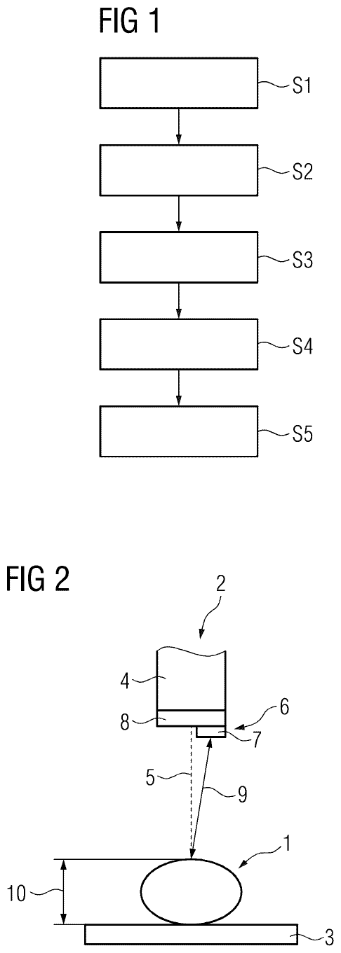

FIG. 1 shows a general flow chart of an exemplary embodiment of the method according to the present invention, wherein specific applications are then to be explained with regard to FIGS. 2 to 6.

Herein, in step S1, a tomosynthesis process is performed with an X-ray device as an imaging process in which a plurality of projection images of an examination object are captured in different capturing geometries by moving at least one X-ray emitter of an X-ray device. Here, cone beam geometry is used so that even with parallel movement of the X-ray emitter and an associated X-ray detector, different directions of radiation are available for voxels of interest, which allow three-dimensional information, in particular depth information, to be derived.

In step S2, measurement data is captured with at least one measuring device and/or means that detects the examination object when the examination object, here a patient, has been positioned, i.e., before and/or during and/or after the capturing of the projection images in step S1.

In step S3, the measurement data is evaluated in order to ascertain, at least partially, extension information of the examination object in at least one spatial direction.

In step S4, the extension information is used to parameterize a reconstruction process to obtain a final image dataset and therefore to determine at least one reconstruction parameter in dependence on the extension information.

In step S5, the image dataset is then reconstructed from the projection images with the at least one reconstruction parameter determined.

In the following exemplary embodiments, the spatial direction in which the extension is described by the extension information corresponds to a direction described by the central beam of the X-ray emitter in at least one of the projection geometries, for example in a central or otherwise identified and/or predetermined projection geometry. For example, the spatial direction can correspond to a slice direction in which tomosynthesis slice images of the image dataset follow one another; in another case, this may entail a direction in which a focal area for the reconstruction of a slice is defined, in particular with an extended field of view, i.e., perpendicular to this slice.

Herein, for the sake of simplicity, in the following examples, corresponding components are designated with the same reference symbols, even when components with different embodiments are used.

FIG. 2 shows a first example of the positioning of a portion of an examination object 1 to be captured, which can, for example, be used in mammography in a mammography device as an X-ray device 2. Herein, in the present case, the examination object 1 is placed on the X-ray detector 3, or more precisely on a cover of the X-ray detector 3. In the present case, a compression plate is not used, so that the distance from the X-ray emitter 4 to the boundary of the examination object 1 along the central beam 5 as the spatial direction is ascertained by using a measuring device and/or means 6, in the present case a camera 7 measuring in three dimensions, which is attached to a collimator device 8 of the X-ray emitter 4 and registered with the coordinate system of the X-ray device 2. The camera measuring in three dimensions that is used can, for example, be a terahertz camera, a TOF camera and/or a stereoscopic camera; as an alternative to a camera measuring in three dimensions, it is also possible to use another distance sensor, for example a radar sensor. It is evident that the measuring device 6 is directed at the examination object 1, wherein, due to the known arrangement of the measuring device 6, a measured distance 9 can be easily converted into a distance between the X-ray emitter 4, or more precisely its X-ray focus, and, in this case, the upper boundary of the examination object 1. The lower boundary of the extension 10 of the examination object 1 is easily obtained in another way from the position of the upper side of the examination object 1 resting on the X-ray detector 3 without the measurement data of the measuring device 6 since the position of the X-ray detector 3 in a control device controlling the operation of the X-ray device 2 is known.

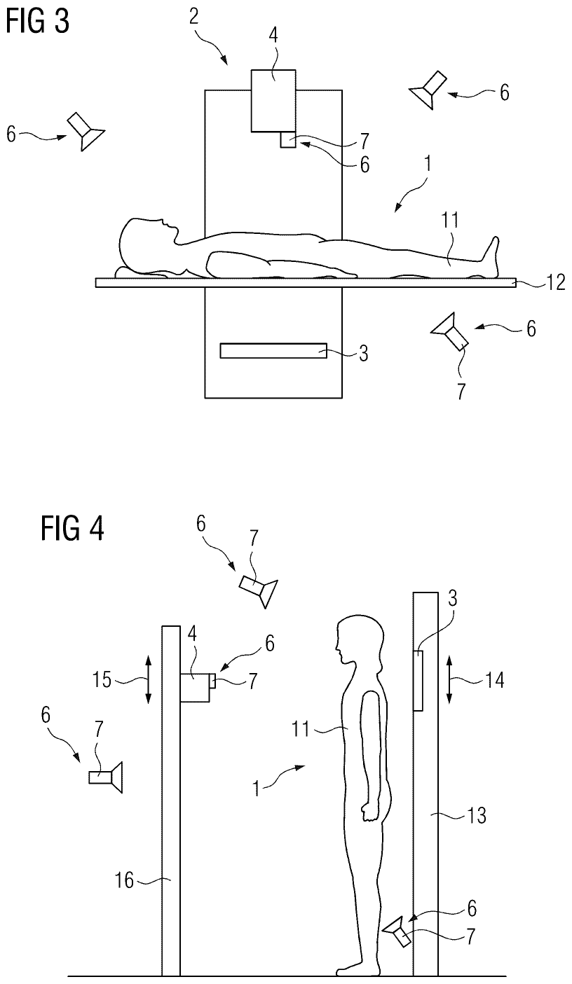

In the second example in FIG. 3, the examination object 1, here the entire patient 11, is arranged lying on a patient bench 12 of the X-ray device 2, which, in the present case, is embodied as a radiography and fluoroscopy device. Since it may also be conceivable to use capturing in which the corresponding portion of the patient 11 is not lying on the patient bench 12, for example when capturing the knee with the knee bent, in addition to the measuring device 6 arranged on the X-ray emitter 4, further measuring device 6 are arranged at various positions, which are known in the coordinate system of the X-ray device 2, in the present case once again cameras 7 measuring in three dimensions.

The measurement data from the various distributed measuring device 6, here cameras 7, can be used in combination to determine a three-dimensional model of the examination object 1, or more precisely its surface profile, in the coordinate system of the X-ray device 2. This makes it possible to ascertain extension information for various positions and spatial directions that are covered in the three-dimensional model. Obviously, this also applies to other examples, in which a plurality of measuring device 6, in particular cameras 7 measuring in three dimensions, are used.

Herein, it should also be noted at this point that it is in principle also conceivable to use manually operated measuring device 6, for example measuring tapes, in particular on the X-ray emitter 4 (or the collimator device 8). Corresponding manually ascertained measurement data can then be entered via an input device of the X-ray device 2, for example a touch user interface.

FIG. 4 shows a further example for positioning the examination object 1, here once again a patient 11, in the X-ray device 2, which is in turn embodied as a radiography and fluoroscopy device. In the present case, the patient 11 is arranged standing in front of a detector wall 13 in which the X-ray detector 3 can be moved in a capturing direction according to the arrow 14. Accordingly, see arrow 15, the X-ray emitter 4 is arranged on a stand 16 according to the arrow 15 so that it can be displaced in the capturing direction so that parallel displacement of the X-ray emitter 4 and the X-ray detector 3 enables larger areas of the patient 11 to be scanned, for example the head and torso thereof and/or at least one leg of the patient 11. Here, it is in particular possible for a so-called slot scan to be performed, in which, for example, a collimator device 8 significantly reduces the detection area in the capturing direction. Nevertheless, cone beam geometry remains in which fluoroscopy of voxels takes place in different directions.

In the third example in FIG. 4, in addition to the measuring device 6 arranged on the X-ray emitter 4 (or the collimator device 8), further measuring device 6 embodied as cameras 7 measuring in three dimensions are also distributed in the room such that the patient 11 can be modeled as completely as possible in three dimensions, in particular it is also possible to identify its distance from the detector wall 13. In particular, the central beam direction perpendicular to the capturing direction at least one position, in particular a plurality of positions, of the capturing arrangement consisting of the X-ray emitter 4 and X-ray detector 3 is to be used as the spatial direction for the extension information.

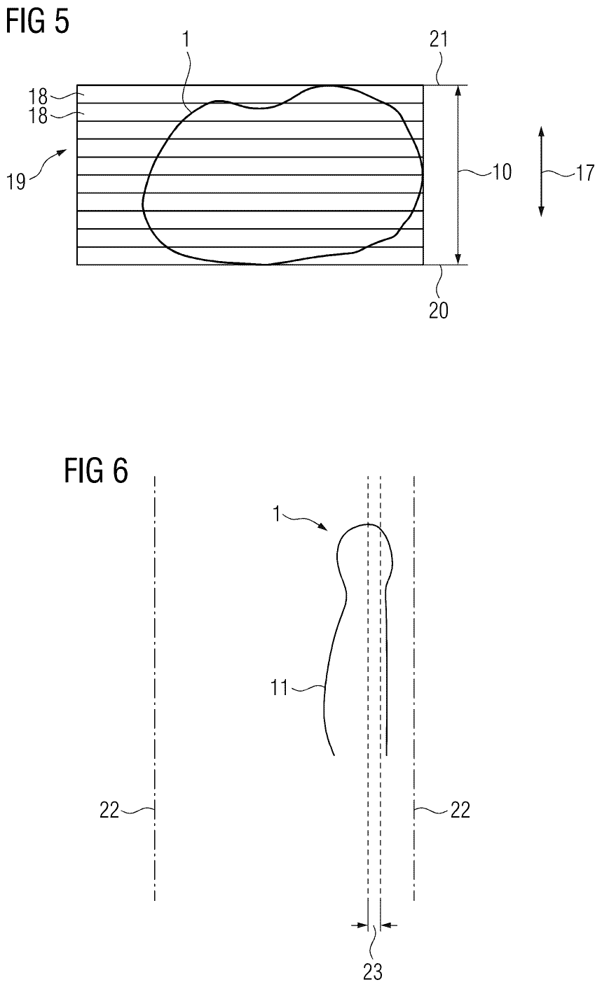

FIG. 5 shows a first application of the extension information in the spatial direction 17, which here corresponds to a slice direction, for restricting the reconstruction area in the slice direction/spatial direction 17 in such a way that the examination object 1 is covered as precisely as possible, but completely, in the slices or slice images 18 of the slice stack 19 in the spatial direction 17. For this purpose, slices are defined over the entire extension 10 of the examination object 1 according to the extension information, i.e., from the lower boundary 20 in FIG. 5 to the upper boundary 21 in FIG. 5, for which the slice images 18 covering the entire examination object 1 are then ascertained.

FIG. 6 shows a second application of the extension information in the case of a slot scan in which the X-ray emitter 4 and the X-ray detector 3 move on parallel trajectories 22 in order to cover a larger area of the patient 11 as the examination object 1. From the extension information, the extension of the patient 11 perpendicular to the trajectories 22 in the capturing direction is known to the coordinate system of the X-ray device so that, together with position information on the position of a structure of interest, in this example the spine, within the patient for a single slice image of the image dataset, the area in which this is in sharp focus, i.e., the focal area 23, can be determined extremely precisely. Here, therefore, the extension information is used to define the focal area 23 such that the structure of interest is depicted in as sharp a focus as possible.

In particular, the slice image showing the focal area 23 in sharp focus over the entire image area on the patient 11, preferably exactly one, can also be ascertained to reflect the real size (“true to scale”), to which in particular the depth information of the tomosynthesis process also contributes.

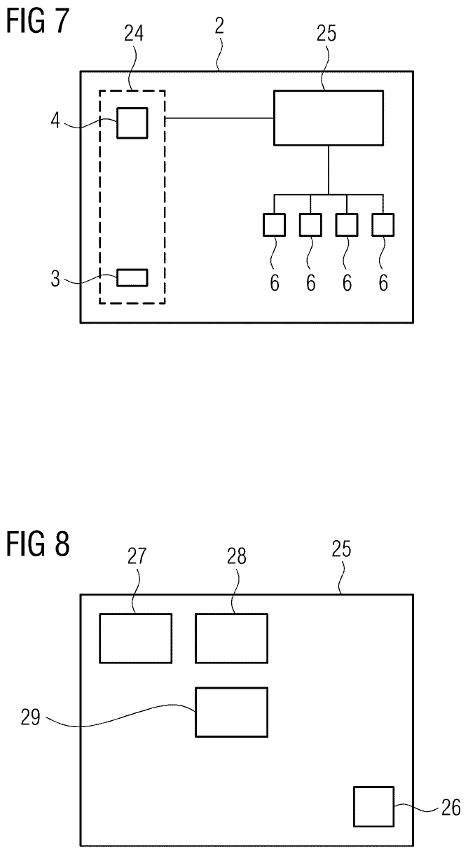

FIG. 7 shows a schematic functional depiction of a general exemplary embodiment of an X-ray device 2 according to the present invention. As already explained, this comprises a capturing arrangement 24 with the X-ray emitter 4 and the X-ray detector 3. The operation of the X-ray device 2 is controlled via a control device 25, which, in the present case, also receives the measurement data from the measuring device 6.

FIG. 8 shows the functional structure of the control device 25 in more detail. In addition to a memory device and/or means 26, in the present case, this initially comprises a capturing unit 27 that controls the capturing operation of the X-ray device 2 and is also embodied to perform step S1. In addition to capturing the projection images, the capturing unit 27 can also control the capturing activity of the measuring device 6, i.e., perform step S2 in coordination with step S1.

In an ascertaining unit 28, the extension information is ascertained in the coordinate system of the X-ray device 2 according to step S3. The extension information is then evaluated by a reconstruction unit 29 for the parameterization of the reconstruction process (step S4), wherein the reconstruction unit 29 also performs the reconstruction process according to step S5 in order to ascertain the image dataset.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been illustrated and described in more detail by the preferred exemplary embodiment, the present invention is not restricted by the disclosed examples and other variations can be derived herefrom by the person skilled in the art without departing from the scope of protection of the present invention.

What is claimed is:

1. A computer-implemented method for operating an X-ray device during an imaging process on an examination object, wherein an image dataset covering a reconstruction area is reconstructed from projection images of the examination object, wherein the projection images are captured in different projection geometries of an X-ray emitter relative to an X-ray detector of the X-ray device, and wherein the computer-implemented method comprises:
ascertaining extension information from measurement data of at least one measuring device, the extension information describing an extension of the examination object in at least one spatial direction; and
parameterizing a reconstruction process based on the extension information.

2. The computer-implemented method as claimed in claim 1, wherein
the imaging process is a tomosynthesis process, or
the different projection geometries are established either by parallel displacement of the X-ray emitter and the X-ray detector or by movement of the X-ray emitter relative to the X-ray detector, wherein the X-ray detector is fixed.

3. The computer-implemented method as claimed in claim 1, wherein the X-ray device is a mammography device or at least one of a radiography or fluoroscopy device.

4. The computer-implemented method as claimed in claim 1, wherein the measuring device includes at least one of at least one distance sensor or at least one camera measuring in three dimensions.

5. The computer-implemented method as claimed in claim 4, wherein at least one of the at least one distance sensor or the at least one camera is fastened to the X-ray emitter or to a collimator device, which is connected to the X-ray emitter.

6. The computer-implemented method as claimed in claim 4, wherein at least one of (i) the at least one distance sensor includes a plurality of distance sensors or (ii) the at least one camera includes a plurality of cameras, wherein at least one of the plurality of distance sensors or the plurality of cameras are arranged at different positions, and wherein the measurement data is evaluated at least in the spatial direction to form a model describing boundaries of the examination object.

7. The computer-implemented method as claimed in claim 1, wherein at least one of a known position or a position detectable via at least one of the at least one measuring device of an examination object-side boundary of a component of the X-ray device is used as at least one boundary of the extension of the examination object in the spatial direction when the examination object is mounted on the component of the X-ray device.

8. The computer-implemented method as claimed in claim 1, wherein the reconstruction area in the spatial direction is restricted to an extent of the examination object.

9. The computer-implemented method as claimed in claim 8, wherein at least one of
the spatial direction is a slice direction, or
the spatial direction is a direction of a central beam of the X-ray emitter, for at least one projection geometry.

10. The computer-implemented method as claimed in claim 1, further comprising:
capturing an image area, which is extended with respect to a detection area of the X-ray device, in a capturing direction perpendicular to the spatial direction by a parallel movement of the X-ray emitter and the X-ray detector in the capturing direction; and
selecting a focal area of the reconstruction process based on the extension information.

11. The computer-implemented method as claimed in claim 10, wherein the projection images are captured as a slot scan.

12. The computer-implemented method as claimed in claim 10, wherein the focal area is selected based on a position of a structure to be mapped in the spatial direction in the examination object.

13. An X-ray device comprising:
an X-ray emitter;
an X-ray detector;
at least one measuring device; and
a control device configured to execute the computer-implemented method as claimed in claim 1.

14. An non-transitory computer-readable medium storing computer-executable instructions that, when executed by at least one processor at an X-ray device, cause the X-ray device to perform the computer-implemented method of claim 1.

15. The computer-implemented method as claimed in claim 4, wherein at least one of the at least one distance sensor or the at least one camera is fastened to the X-ray emitter or to a component of the X-ray device, which is connected to the X-ray emitter.

16. The computer-implemented method as claimed in claim 10, wherein the projection images are captured with collimation in the capturing direction.

17. The computer-implemented method as claimed in claim 6, wherein at least one of a known position or a position detectable via at least one of the at least one measuring device of an examination object-side boundary of a component of the X-ray device is used as at least one boundary of the extension of the examination object in the spatial direction when the examination object is mounted on the component of the X-ray device.

18. The computer-implemented method as claimed in claim 17, further comprising:
capturing an image area, which is extended with respect to a detection area of the X-ray device, in a capturing direction perpendicular to the spatial direction by a parallel movement of the X-ray emitter and the X-ray detector in the capturing direction; and
selecting a focal area of the reconstruction process based on the extension information.

19. The computer-implemented method as claimed in claim 6, wherein the reconstruction area in the spatial direction is restricted to an extent of the examination object.

20. The computer-implemented method as claimed in claim 6, further comprising:
capturing an image area, which is extended with respect to a detection area of the X-ray device, in a capturing direction perpendicular to the spatial direction by a parallel movement of the X-ray emitter and the X-ray detector in the capturing direction; and
selecting a focal area of the reconstruction process based on the extension information.
